(19) United States
(12) Patent Application Publication
(10) Pub. No.: US 2022/0233730 A1
(43) Pub. Date: Jul. 28, 2022

(54) SANITIZATION USING ULTRAVIOLET LIGHT WITH IMAGE CAPTURE DEVICE

(71) Applicant: QUALCOMM Incorporated, San Diego, CA (US)

(72) Inventors: Suresh Kumar BITRA, Hyderabad (IN); Naga Chandan Babu GUDIVADA, Hyderabad (IN); Rakesh PALLERLA, Hyderabad (IN); Prakash TIWARI, Hyderabad (IN)

(21) Appl. No.: 17/248,517

(22) Filed: Jan. 28, 2021

(51) Int. Cl. A61L 2/10; G06T 7/11; G06T 7/521; H04N 5/225; H04M 1/02; G01B 11/22; G01S 17/08

(52) U.S. Cl. CPC A61L 2/10; G06T 7/11; G06T 7/521; H04N 5/2256; G06K 9/00375; G01B 11/22; G01S 17/08; G06T 2207/30196; H04M 1/0264

(57) ABSTRACT

An electronic device with a camera uses UV light to sanitize objects such as surfaces or hands. An object is identified by the electronic and placed at an optimal distance from the electronic device. At least part of the object is exposed to UV light emitted from the electronic device for a sufficient duration to achieve disinfection. In some implementations, instructions are delivered to a user associated with the electronic device to position the object so that a remainder of the object is exposed to UV light for disinfection. The electronic device may be moved relative to the object to continue disinfection. In some implementations, an indication is provided of the status of disinfecting the object.

### TECHNICAL FIELD

This disclosure relates generally to disinfection of objects and human body parts and more particularly to disinfection of objects and human body parts using ultraviolet (UV) light with electronic devices such as smart devices.

### DESCRIPTION OF RELATED TECHNOLOGY

The surfaces of objects tend to attract and harbor potentially harmful organisms such as microbes, pathogens, viruses, bacteria, and the like. Human body parts such as the hands tend to attract and harbor such potentially harmful organisms encountered through day-to-day activities. Public awareness has increased over the years regarding how germs are spread that lead to illnesses such as influenza, norovirus infection, Middle East Respiratory Syndrome (MERS), Ebola, Zika, and Covid-19. More precautions are being taken to sterilize an environment against pathogens and to frequently sanitize hands to limit the spread of infectious diseases.

Disinfection of objects can be accomplished using a various soaps, sprays, sanitizing gels, and disinfectant wipes. However, it is often inconvenient to carry around bulky wipes, bottles of disinfectant, and hand sanitizers. Not only is it cumbersome to carry around bottles and wipes, but traditional methods of sanitization generally require personal contact and can contain toxic ingredients. Traditional methods of sanitization may also leave an undesirable residue and generate more waste for the environment. UV radiation has been discovered to effectively destroy microorganisms and has been used in sanitizing and disinfecting surfaces in various places such as homes, hospitals, cars, and businesses. Technologies for applying UV light for disinfection have largely been limited to stationary objects and can be dangerous to humans.

### SUMMARY

The devices, systems, and methods of this disclosure each have several aspects, no single one of which is solely responsible for the desirable attributes disclosed herein.

One aspect of the subject matter of this disclosure can be implemented in an electronic device. The electronic device includes an imaging source, a UV light source, and a control system communicatively connected to the imaging source and the UV light source. The control system is configured to: identify an object for disinfection using the imaging source, expose at least a first portion of the object to UV light from the UV light source, wherein the object is at a desired distance from the UV light source, and determine that the object has been disinfected.

In some implementations, the electronic device further includes a display, where the imaging source is configured to display an image showing the object to be disinfected in the display. In some implementations, the control system is further configured to segment the image containing the object into a plurality of segments, each segment corresponding to different portions of the object, and expose each portion of the object corresponding to the plurality of segments to UV light for a sufficient duration to complete disinfection of the object. In some implementations, the UV light source is configured to emit far UVC light. In some implementations, the desired distance is calculated based at least in part on an intensity of the UV light, a wavelength of the UV light, and desired level of disinfection in at least the first portion of the object. In some implementations, the control system is further configured to: instruct a user associated with the electronic device to place the object relative to the imaging source so that at least a second portion of the object is positioned to be exposed to the UV light source, and expose at least the second portion of the object to UV light from the UV light source. In some implementations, the control system is further configured to: provide an indication to a user via visual, auditory, or haptic feedback of how much the object has been disinfected.

Another innovative aspect of the subject matter described in this disclosure can be implemented in a method for disinfecting an object. The method includes identifying an object for disinfection using a camera of an electronic device, where the electronic device includes the camera and a UV light source, exposing at least a first portion of the object to UV light from the UV light source, where the object is at a desired distance from the UV light source, and determining that the object has been disinfected.

In some implementations, the method further includes instructing a user associated with the electronic device to place the object relative to the camera so that at least a second portion is positioned to be exposed to the UV light source, and exposing at least the second portion of the object to UV light from the UV light source. In some implementations, the method further includes providing an indication to a user via visual, auditory, or haptic feedback of how much the object has been disinfected. In some implementations, exposing at least the first portion of the UV light includes exposing at least the first portion of the object to UV light for a specified duration to deliver a desired level of UV dose. In some implementations, the electronic device further includes a display for displaying an image showing the object to be disinfected, where the method further includes segmenting an image containing the object into a plurality of segments, each segment corresponding to different portions of the object, and exposing each portion of the object corresponding to the plurality of segments to UV light for a sufficient duration to complete disinfection of the object.

### BRIEF DESCRIPTION OF THE DRAWINGS

Details of one or more implementations of the subject matter described in this specification are set forth in the accompanying drawings and the description below. Other features, aspects, and advantages will become apparent from the description, drawings and claims. Note that the relative dimensions of the following figures may not be drawn to scale.

Like reference numbers and designations in the various drawings indicate like elements.

FIG. 1 shows a block diagram representation of components of an example electronic device that includes an imaging source and a UV light source according to some implementations.

FIG. 2 shows a cross-sectional schematic representation of an imaging source and a UV light source on a printed circuit board (PCB) incorporated in an example electronic device according to some implementations.

FIG. 3 shows a perspective view of a schematic illustration of an example electronic device including an imaging source and a UV light source for disinfecting an object according to some implementations.

FIG. 4 shows a flow diagram illustrating an example process for disinfecting an object according to some implementations.

FIGS. 5A-5E show an image capture device across various stages of an example process for disinfecting an object according to some implementations.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Various embodiments will be described in detail with reference to the accompanying drawings. References made to particular examples and implementations are for illustrative purposes, and are not intended to limit the scope of the claims.

UV radiation has been used effectively in various applications to disinfect and sanitize hospital rooms, medical clinics, food production facilities, and drinking water. UV radiation has been used effectively to disinfect and sanitize toothbrushes, shoes, mattresses, keyboards, faucets, and kitchenware. UV radiation has used with heating, ventilation, and air conditioning (HVAC) systems or air purifiers to disinfect the air.

It can be cumbersome and difficult to always carry around hand sanitizers or disinfectant wipes. However, many people carry around portable electronic devices such as mobile phones, making such devices easily accessible in the daily activities of life. With increasing demands to keep areas and people sanitized, challenges exist to converting portable electronic devices such as mobile phones into safe and effective sanitizers.

Additionally, it can be cumbersome and time-consuming to disinfect fields, crops, roads, and other common places. However, an electronic device such as a drone may be controlled or programmed to sanitize large surface areas. Challenges exist to converting electronic devices such as drones into safe and effective cleaning agents.

The present disclosure relates to an electronic device that includes a UV light source and an imaging source, where the electronic device is useful for disinfection of objects. The electronic device may be a portable electronic device such as a smartphone. The imaging source may be a camera. The electronic device identifies an object such as a hand. The object is placed at a desired distance from the electronic device. The electronic device exposes at least a portion of the object to UV radiation from the UV light source. The electronic device may provide instructions to a user so that a remainder of the object is exposed to UV radiation. The electronic device may provide an indication to user when the object is successfully disinfected.

Particular implementations of the subject matter described in this disclosure may be implemented to realize one or more of the following potential advantages. Combining the disinfection source and the electronic device facilitates convenience and ease of use for sanitization. Instead of wipes or sanitizing gels, the electronic device uses UV radiation that can be recycled, does not contain toxic chemicals, and does not lead to waste. The electronic device can use the imaging source to identify an object for disinfection, determine safe or optimal distances, and select a suitable UV wavelength, UV intensity, and duration of exposure, especially if the object is a human body part. The electronic device can also use at least the imaging source to determine how much of the object is exposed to UV radiation and assist a user in moving the object or electronic device so that the entirety of the object can be disinfected. In some implementations, the electronic device provides visual, audio, or haptic feedback to assist user navigation when disinfecting the object. In some implementations, the electronic device uses a flashlight to visually present a targeted area of exposure to the user. In some implementations, the electronic device segments an image of the object into a plurality of segments each corresponding to an object area of the object. The UV light source may be configured to emit UV radiation at a beam coverage area to expose an object area corresponding to a segment. This can add to visual appeal for a user interface and facilitate advancement of disinfection sequentially.

As used herein, the term “object” may be used to describe any inanimate object or animate object. Accordingly, an “object” in the present disclosure is inclusive of body parts such as hands, feet, torso, etc. As used herein, the term “imaging source” describes any device or system with image capture capabilities. Accordingly, an “imaging source” in the present disclosure is inclusive of cameras such as digital cameras or thermal imaging cameras.

The implementations of the present disclosure may be implemented in any device, apparatus, or system that includes an imaging source or image capture device such as a camera. The described implementations may be included in or associated with a variety of electronic devices such as, but not limited to: mobile phones, smartphones, drones, wearable devices such as bracelets, armbands, wristbands, rings, headbands and patches, etc., hand-held or portable computers, laptops, notebooks, tablets, cameras, game consoles, clocks, calculators, monitors, flat panel displays, electronic reading devices (e.g., e-readers), or other devices with a built-in camera. Thus, the teachings are not intended to be limited to the implementations depicted solely in the Figures, but instead have wide applicability as will be readily apparent to one having ordinary skill in the art.

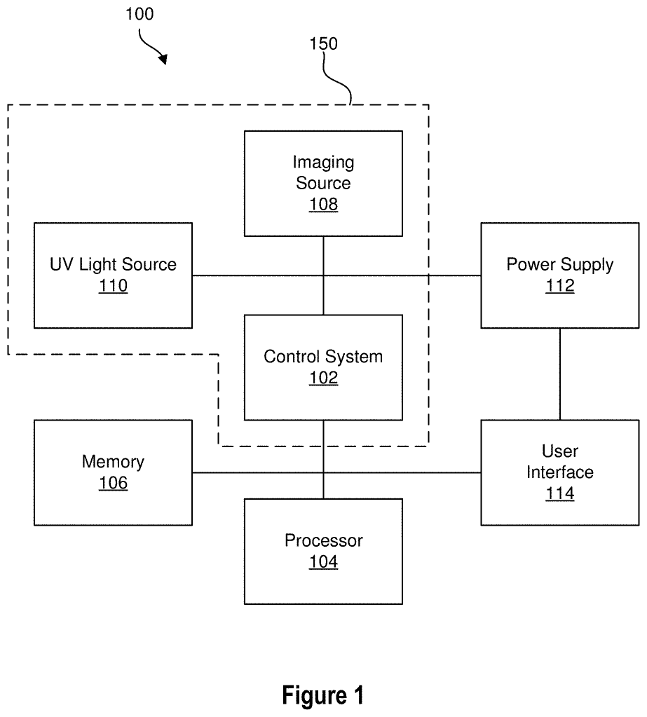

FIG. 1 shows a block diagram representation of components of an example electronic device that includes an imaging source and a UV light source according to some implementations. The electronic device 100 may be representative of, for example, various portable computing devices such as cellular phones, smartphones, smart watches, drones, multimedia devices, personal gaming devices, tablet computers and laptop computers, among other types of portable computing devices. However, various implementations described herein are not limited in application to portable computing devices. Indeed, various techniques and principles disclosed herein may be applied in traditionally non-portable systems and devices, such as in computer monitors, television displays, among other applications. Additionally, various implementations described herein are not necessarily limited in application to devices that include displays.

As shown in FIG. 1, the electronic device 100 includes a control system 102, a processor 104, a memory 106, an imaging source 108, a UV light source 110, a power supply 112, and an interface 114. The control system 102 may also be referred to as a controller or system controller. While the control system 102 is shown and described as a single component, in some implementations, the control system 102 may collectively refer to two or more distinct control units or processing units in electrical communication with one another. In some implementations, the control system 102 may include one or more of a general purpose single- or multi-chip processor, a central processing unit (CPU), a digital signal processor (DSP), an applications processor, an application specific integrated circuit (ASIC), a field programmable gate array (FPGA) or other programmable logic device (PLD), discrete gate or transistor logic, discrete hardware components, or any combination thereof designed to perform the functions and operations described therein.

In addition, the electronic device 100 of FIG. 1 may include a processor 104 and a memory 106. In some implementations, the processor 104 communicates data to the control system 102 including, for example, instructions or commands. In some such implementations, the control system 102 may communicate data to the processor 104 including, for example, raw or processed image data, location or depth data, orientation data, user input data, or other types of information to be processed by the processor 104. It should be understood that, in some other implementations, the functionality of the control system 102 may be implemented entirely, or at least partially, by the processor 104. In some such implementations, a separate control system 102 may not be required because the function of the control system 102 may be performed by the processor 104 of the electronic device 100.

Depending on the implementation, one or both of the control system 102 and the processor 104 may store data in the memory 106. By way of an example, the data stored in the memory 106 may include raw image data, filtered or otherwise processed image data, estimated image data, or final refined image data. The memory 106 may store data associated with the UV light source 110. Such data may include optimal distances between the UV light source 110 and an identified object (e.g., hand) based on a wavelength of the UV light source 110. Such data may additionally or alternatively include UV dosage (mJ/cm²) based on a wavelength of the UV light source 110 for achieving certain levels of disinfection. The memory 106 may store data associated with detection of objects from image data provided by the imaging source 108. This can include machine learning algorithms or other image processing methods. The memory 106 may store data regarding objects that have been sanitized, how much was sanitized, and when such objects were sanitized. The memory 106 may store processor-executable code of other executable computer-readable instructions capable of execution by one or both of the control system 102 and the processor 104 to perform various operations (or to cause other components such as the imaging source 108, UV light source 110, and/or sensors to perform operations), including any of the calculations, computations, estimations, or other determinations described herein. It should also be understood that the memory 106 may collectively refer to one or more memory devices (or “components”). For example, depending on the implementation, the control system 102 may have access to and store data in a different memory device than the processor 104. In some implementations, one or more of the memory components may be implemented as a NOR- or NAND-based flash memory array. In some other implementations, one or more of the memory components may be implemented as a different type of non-volatile memory. Additionally, in some implementations, one or more of the memory components may include a volatile memory array such as, for example, a type of RAM.

In some implementations, the control system 102 or the processor 104 may communicate data stored in the memory 106 or data received directly from the imaging source 108 or other sensor through an interface 114. For example, such communicated data can include image data or data derived or otherwise determined from image data. The interface 114 may collectively refer to one or more interfaces of one or more various types. In some implementations, the interface 114 may include a memory interface for receiving data from or storing data to an external memory such as a removable memory device. Additionally or alternatively, the interface 114 may include one or more wireless network interfaces enabling transfer of raw or processed data to, as well as the reception of data from, an external computing device, system, or server.

A power supply 112 may provide power to some or all of the components in the electronic device 100. The power supply 112 may include one or more of a variety of energy storage devices. For example, the power supply 112 may include a rechargeable battery, such as a nickel-cadmium battery or lithium-ion battery. Additionally or alternatively, the power supply 112 may include one or more supercapacitors. In some implementations, the power supply 112 may be chargeable (or “rechargeable”) using power accessed from, for example, a wall socket (or “outlet”) or a photovoltaic device (or “solar cell” or “solar cell array”) integrated with the electronic device 100. Additionally or alternatively, the power supply 112 may include a power management integrated circuit and a power management system.

The electronic device 100 includes a sanitization system 150 that implements various internal components or sensors of the electronic device 100 for disinfecting an object external to the electronic device 100. The sanitization system 150 may include at least the control system 102, the imaging source 108, and the UV light source 110. However, it will be understood that the other components of the electronic device 100 such as the processor 104, the memory 106, the power supply 112, and the interface 114 may assist in executing the operations of the sanitization system 150.

The imaging source 108, the UV light source 110, and the control system 102 may each be embedded in the electronic device 100. In some implementations, the imaging source 108 may be any device configured to capture images, either continuously or intermittently. The imaging source 108 may be configured to provide raw or processed image data to the control system 102. The imaging source 108 may include a camera such as a digital camera or thermal imaging camera, machine vision, and/or laser. In some implementations, the imaging source 108 is a camera. In some implementations, the camera may include a lens, an image sensor for converting an object image to electrical image signals, an image processor for processing incoming image signals into frames of pixels, an optical image stabilization or auto-focus actuator coupled to the lens, and a camera controller, among other camera components. The imaging source 108 may provide visual information to the control system 102 regarding an object external to the electronic device 100. In some implementations, the imaging source 108 includes or is coupled to a depth sensor to determine a distance from the object external to the electronic device 100.

The UV light source 110 emits UV light at one or more wavelengths in the range of 10 nm to 400 nm. In some implementations, the UV light source 110 emits UV light as ultraviolet C having a wavelength in the range of 100 nm to 280 nm, such as about 254 nm. Such wavelengths are known to be effective in destroying, killing, or retarding growth of infectious agents and other microorganisms. Thus, UV light is useful for disinfecting, sanitizing, and/or sterilizing objects. In some implementations, the UV light source 110 is configured to emit a wavelength between about 207 nm and about 222 nm, such as about 220 nm. Such wavelengths have only a short range in biological material so that it cannot penetrate the dead-cell layer at the surface of human skin and it cannot penetrate the tear layer of the eye. However, such wavelengths of light are still effective in destroying, killing, or retarding growth of infectious agents and other microorganisms. Wavelength ranges of 207-220 nm or 207-222 nm may be referred to as far UVC light. These wavelengths may be deemed safe for human exposure.

In some implementations, the UV light source 110 includes an UV LED or UV LED array. In some implementations, the UV light source 110 includes a fluorescent UV bulb or UV laser. The intensity level of the UV light source 110 may be controlled by adjusting the driving power supplied to the UV light source 110. In some implementations, the UV light source 110 may include or may be coupled to a UV sensor to assist in adjusting the intensity level. In some implementations, the wavelength of UV radiation emitted by the UV light source 110 may be tuned according to the object being disinfected. The control system 102 may specify the wavelength, intensity, and exposure time for exposing the object to UV light from the UV light source 110. The UV light source 110 may be powered by the power supply 112.

The control system 102 is communicatively connected to the imaging source 108 and the UV light source 110. As used herein, “communicatively connected” or “communicatively coupled” may describe devices that are in communication with one another such that signals can be transmitted and/or received between devices. The imaging source 108 may provide image data to the control system 102, and the control system 102 may provide instructions or commands to the UV light source 110 based at least in part on the image data provided by the imaging source 108. For instance, the control system 102 may provide instructions or commands regarding operation of the UV light source 110 based on the image data, such as whether to activate or deactivate the UV light source 110, the intensity of the UV radiation, the wavelength of UV radiation, the exposure time of the UV radiation, etc. The imaging source 108 may provide image data to the control system 102, and the control system 102 may provide instructions or commands to a user associated with electronic device 100. For example, the control system 102 may provide instructions through a display based on the image data for guiding the user in moving and positioning the electronic device 100. Alternatively, the control system 102 may provide instructions via haptic or auditory feedback. The control system 102 processes and provides data to and from the imaging source 108 and the UV light source 110 so that the control system 102 coordinates functions between the imaging source 108 and the UV light source 110. The control system 102 integrates the functions of the imaging source 108 and the UV light source 110 in the sanitization system 150.

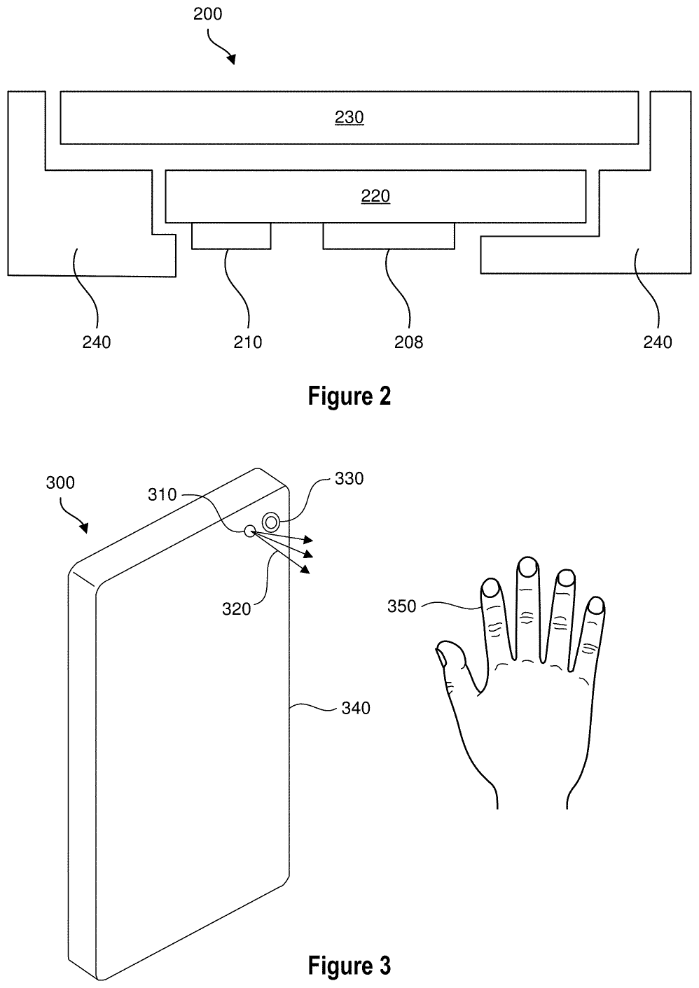

FIG. 2 shows a cross-sectional schematic representation of an imaging source and a UV light source on a printed circuit board (PCB) incorporated in an example electronic device according to some implementations. An electronic device 200 generally includes an enclosure or housing 240 within which various circuits, sensors, and other electrical components reside. In the illustrated example implementation, the electronic device 200 further includes a display 230. The display 230 may be representative of any of a variety of suitable display types such as a digital micro-shutter (DMS)-based display, light-emitting diode (LED) display, an organic LED (OLED) display, a liquid crystal display (LCD), an LCD display that uses LEDs as backlights, a plasma display, an interferometric modulator (IMOD)-based display, or another type of display for displaying an image. In some implementations, the display 230 is a touch-sensitive display.

The electronic device 200 may further include a printed circuit board 220 within the housing 240. A UV light source 210 may be mounted on the printed circuit board 220. In some implementations, the imaging source 208 may also be mounted on the printed circuit board 220. Accordingly, the UV light source 210 and the imaging source 208 may be formed on a common substrate. In some implementations, the UV light source 210 may be proximate the imaging source 208. The UV light source 210 may be communicatively connected to the imaging source 208 via circuitry associated with the printed circuit board 220. Though FIG. 2 only illustrates the UV light source 210 and the imaging source 208 mounted on the printed circuit board 220, it will be understood that other hardware components may be formed on the printed circuit board 220. The printed circuit board 220 may include one or more microprocessors, microcontrollers, field programmable gate arrays, systems-on-a-chip, volatile or non-volatile memory, discrete circuitry, and/or other hardware, software, or firmware. Hardware components such as microprocessors and microcontrollers may facilitate electrical communication between the UV light source 210 and the imaging source 208.

An opening in the housing 240 may allow both the UV light source 210 to transmit UV radiation to objects outside of the housing 240 and the imaging source 208 to capture an image of the objects outside of the housing 240. In some implementations, a window (not shown) is positioned adjacent to the imaging source 208 and/or the UV light source 210 to shield them from the outside environment. The window may be transparent to one or both of UV light and visible light. In some implementations, a cover plate (not shown) is positioned over the display 230 to protect the display 230 and internal components of the electronic device 200 from the outside environment. As illustrated in FIG. 2, the UV light source 210 is mounted on a side of the printed circuit board 220 facing away from the display 230. In other words, the display 230 and the UV light source 210 are on opposite sides of the printed circuit board 220. As a result, a user facing the display 230 can view the object being disinfected by the UV light source 210 on the display 230. Otherwise, the UV light source 210 may undesirably emit UV radiation towards the user.

FIG. 3 shows a perspective view of a schematic illustration of an example electronic device including an imaging source and a UV light source for disinfecting an object according to some implementations. An electronic device in FIG. 3 is a portable electronic device such as a mobile phone 300. The mobile phone 300 may include a housing 340 for enclosing various circuits, sensors, and electrical components in the mobile phone 300. The mobile phone 300 may include a UV light source such as a UV LED 310 configured to emit UV radiation 320. The mobile phone 300 may further include an imaging source such as a camera 330 configured to capture images of an environment outside the mobile phone 300. The UV LED 310 and the camera 330 may be communicatively coupled with one another.

To disinfect an object such as a human hand 350, the mobile phone 300 is positioned to target the human hand 350 so that the human hand 350 is exposed to the UV radiation 320 from the UV LED 310. To assist in positioning the mobile phone 300, the camera 330 captures an image of the human hand 350. The camera 330 may employ various sensors to determine a distance of the human hand 350 from the mobile phone 300. The camera 330 may employ machine learning algorithms or other image processing methods to identify the object as the human hand 350. The camera 330 may employ certain image processing methods to determine an area of the captured image that would be exposed to UV radiation 320. The captured image may be segmented in some implementations based on coverage area of the UV radiation 320. Once the human hand 350 is positioned at an optimal distance from the mobile phone 300, the UV LED 310 exposes the human hand 350 to the UV radiation 320. The mobile phone 300 integrates the functions of the UV LED 310 and the camera 330 for safe and effective disinfection of the human hand 350.

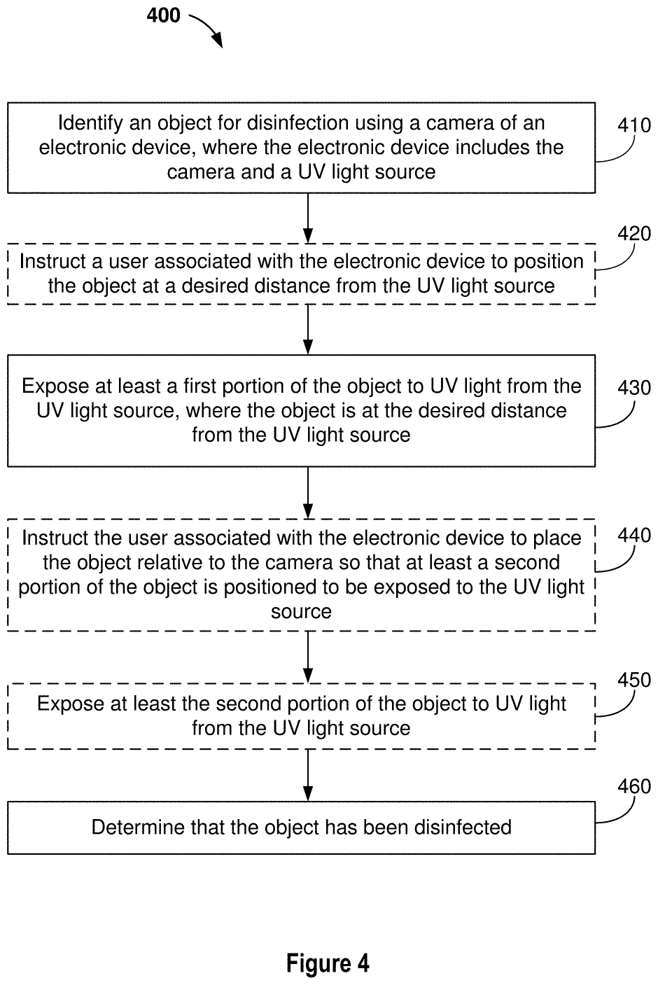

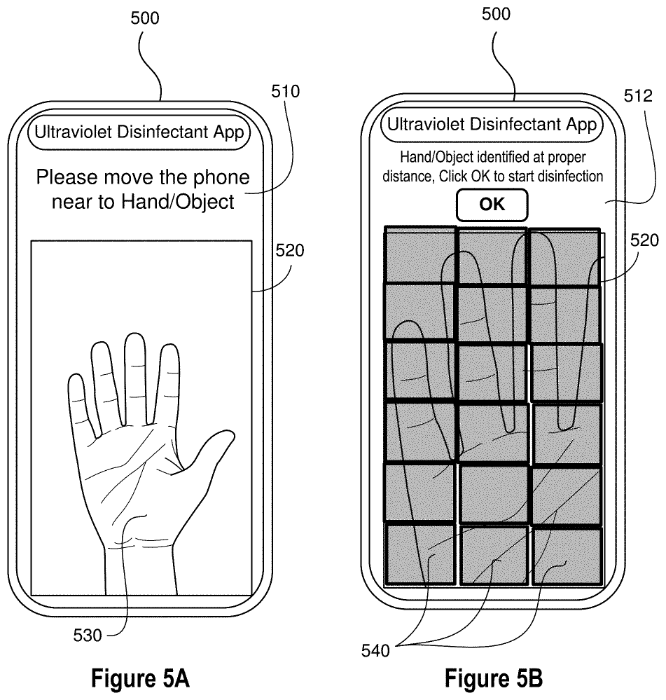

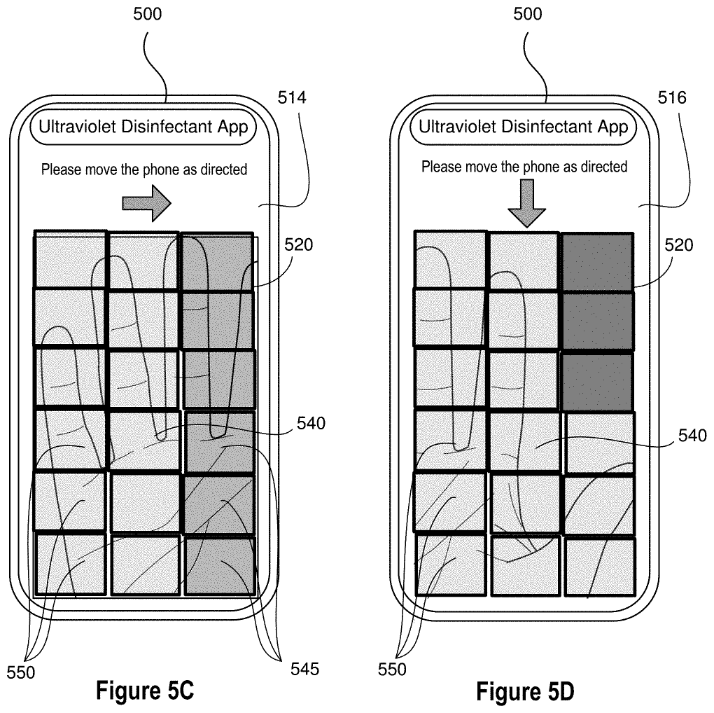

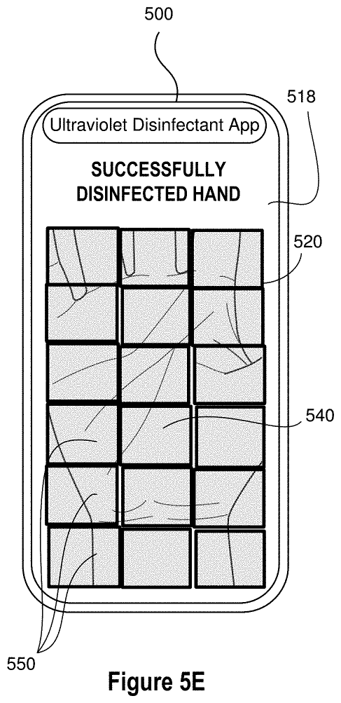

FIG. 4 shows a flow diagram illustrating an example process for disinfecting an object according to some implementations. A process 400 may be performed in a different order or with additional operations. Aspects of the process 400 are described with respect to FIGS. 5A-5E. In some implementations, the operations of the process 400 may be implemented, at least in part, according to software stored in one or more non-transitory computer readable media. In some implementations, the software may be run using an application.

At block 410 of the process 400, an object is identified for disinfection using a camera of an electronic device, where the electronic device includes the camera and a UV light source. The object exists in an environment outside the electronic device. The camera may obtain an image of the environment outside the electronic device, where the image includes at least some or all of the object. The image may be generated using an image sensor associated with the camera. In some implementations, the image may be taken from one or more frames of a video captured by the camera. The image may be received as an input image by a control system, or one or more processors of a control system. Using a machine learning algorithm or other image processing method employed by the control system or one or more processors of the control system, the object may be identified.

By way of an example, object identification may work by determining one or more portions of the input image that include the object. Patterns or salient features of the input image may be recognized within the input image to determine the one or more portions associated with the object. A machine learning model or artificial intelligence is trained to predict an object type based on the input image. Using an appropriate machine learning algorithm, the machine learning algorithm is used to recognize patterns in data points between independent variables (input) and dependent variables (output) so as to accurately predict an object type (new output) when presented with a new input image (new input).

Machine learning algorithms can be divided into three broad categories: supervised learning, unsupervised learning, and reinforcement learning. Supervised learning is useful where a property (label) is available for a certain dataset (training set). Examples of machine learning algorithms that are supervised include but are not limited to linear regression, logistic regression, decision tree, learning vector quantization, support vector machine (SVM), Naive Bayes, k-nearest neighbors, random forest, and gradient boosting. Semi-supervised learning is a type of supervised learning having a small amount of labeled data and a large amount of unlabeled data for a certain dataset. Unsupervised learning is useful where implicit relationships in a given unlabeled dataset (items are not pre-assigned) have not been discovered. An example of a machine learning algorithm that is unsupervised includes k-means. Reinforcement learning falls between supervised and unsupervised learning, where some feedback is available for each predictive step or action but there is no precise label. Rather than being presented with correct input/output pairs as in supervised learning, a given input is mapped to a reward function that an agent is trying to maximize. An example of a machine learning algorithm that is reinforcement-based includes a Markov Decision Process. Other types of learning that may fall into the one or more of the categories described above include, for example, deep learning and artificial neural networks (e.g., convolutional neural networks).

The machine learning algorithm may be trained using a training set. In some implementations, the training set may include a plurality of training set members each having a training image. Training images may generally include images of different object types such as cats, dogs, cars, homes, chairs, tables, cups, roads, plants, hands, feet, eyes, roads, drinking fountains, etc. In some implementations, the training set may be stored in a database accessible by the control system or one or more processors of the control system. In some implementations, the machine learning algorithm is an independently trained inference algorithm or classifier. This means that the inference model is trained separately beforehand, where the independently trained inference algorithm may be trained separately by outside experts, researchers, designers, users, etc. After determining the one or more portions of the input image that includes the object, the machine learning algorithm identifies an object type in the input image. For example, the machine learning algorithm may use deep neural networks to identify the object type from the input image. Accordingly, the control system or one or more processors of the control system may identify that the object is a cat, dog, car, home, chair, table, cup, road, plant, hand, foot, eye, road, drinking fountain, or other object. Recognition of the object may be useful for providing instructions to a user in exposing the object to UV light. Recognition of the object may also be useful for tuning the wavelength, intensity, and/or exposure duration of the UV light when exposing the object to UV light.

Identification of the object may include selection of the object in view of the camera. Multiple objects may be in view of the camera, and the user may select one of the objects for disinfection. In some implementations, identification of the object includes measuring dimensions of the object. Ascertaining the dimensions of the object may be useful for segmenting an image of the object into segments for sanitization and calculating an estimated time for sanitization.

The electronic device may be any portable electronic device with a camera. Such a device may also be referred to as an image capture device. In some implementations, the portable electronic device is a mobile device such as a smartphone or tablet. In some implementations, the portable electronic device is a drone. The electronic device may be equipped with not only a camera, but a UV light source. In some implementations, the UV light source includes one or more UV LEDs or one or more UV lasers. Where the UV light source includes multiple UV LEDs, the multiple UV LEDs may be arranged in an array or panel. In some implementations, the UV light source may include one or more UV LEDs configured to emit a wavelength between about 207 nm and about 222 nm or between about 207 nm and about 220 nm. In some implementations, the electronic device further includes a flashlight that is configured to emit visible light. The electronic device is equipped to provide feedback or instructions to a user. In some implementations, the electronic device may be equipped with a display for visual feedback, lights for visual feedback, a speaker for auditory feedback, and/or vibramotors for haptic feedback. For instance, the electronic device may include a display to provide visual feedback or instructions to the user.

The electronic device may include a control system for integrating functions of the camera and UV light source. The control system may process image data from the camera to identify the object in view of the camera. In some instances, the control system may implement the machine learning algorithm to identify the object in view of the camera.

FIG. 5A shows an image capture device positioned to capture an image of an object. An image capture device 500 may include hardware, software, firmware, or combinations thereof to run an application for objection detection and UV sanitization. In FIGS. 5A-5E, the application may also be referred to as an “ultraviolet disinfectant app.” The application may be a system application or user application. The application may be initiated by user input or automatically under certain conditions. In some implementations, initiation of the application may require user authentication to the image capture device 500. The application may integrate the functions of a camera and a UV light source for object detection and UV sanitization. When the application is initiated, the camera may be automatically activated to capture an image 520 of an area external to the image capture device 500. As used herein, an “image” may refer to a still image or one or more frames of a video.

The image capture device 500 may include a display through which feedback/instructions and an image may be displayed in a user interface. Upon initiating the application, instructions 510 may be provided to the user interface and the image 520 may be displayed. The image 520 may include an object image 530 corresponding to an object in view of the camera. In FIG. 5A, the object image 530 is a human hand. The instructions 510 may request that the user position the image capture device 500 in proximity to the object (or the object in proximity to the image capture device 500) so that the object is in view of the camera.

In some implementations, the object is identified for disinfection using at least the camera of the image capture device 500. The object image 530 may provide an entirety of the object or a portion thereof for disinfection using the application. In some implementations, the application may identify an object type associated with the object image 530 using a machine learning algorithm or other image processing method. Example object types include but are not limited to cats, dogs, cars, homes, chairs, tables, cups, roads, plants, hands, feet, eyes, roads, drinking fountains, etc. Data regarding the object such as object type, amount/percentage disinfected, object image, etc. may be stored in a memory or other database associated with the image capture device 500. In some implementations, the object image 530 may be selected from the image 520 by the user for disinfection.

Returning to FIG. 4, at block 420 of the process 400, a user associated with the electronic device is optionally instructed to position the object at a desired distance from the UV light source. The desired distance may be calculated based at least in part on characteristics of the UV light emitted from the UV light source. Such characteristics may be found in data related to the UV light source, which can be found, for example, in data sheets, product information, or factory settings of the UV light source. Characteristics of the UV light may include but are not limited to peak wavelength, irradiance, scattering or intensity distribution, irradiation pattern, beam width, and viewing angle, among other characteristics. That way, the amount of surface area covered by the UV light and the intensity of the UV light irradiated can be determined at a given distance. When the UV light source is installed or otherwise provided in the electronic device, these characteristics of the UV light are known upon initial factory calibration. Accordingly, the desired distance may be tuned or calibrated at the time the UV light source is integrated in the electronic device. As used herein, the “desired distance” refers to an optimal distance or range of distances to an object for safe and effective disinfection of the object. Accordingly, the “desired distance” may represent a predetermined distance or predetermined range of distances between the object and the UV light source for achieving safe and effective disinfection.

In some implementations, a distance between the object and the UV light source can be ascertained using a depth sensor. Depth sensors may also be referred to as distance sensors, range sensors, or proximity sensors for determining a distance to an object. In some implementations, the camera may be equipped with the depth sensor or the depth sensor may be a separate component in the electronic device. In some implementations, the camera may use time-of-flight (ToF) depth sensing to calculate the distance between the object and the UV light source.

After identifying the object for disinfection, instructions may be provided to the user associated with the electronic device. In some implementations, the instructions may be provided in a user interface of the electronic device. In some implementations, the instructions may be provided as auditory commands from a speaker of the electronic device. The instructions may direct the user to position the object at the desired distance from the electronic device.

The object may be positioned at the desired distance by moving the object relative to the electronic device in view of the camera or moving the electronic device relative to the object in view of the camera. The electronic device may be configured to output instructions to the user for positioning the object at the desired distance. In some implementations, the electronic device may output feedback such as visual, auditory, or haptic feedback to the user when the object is positioned at the desired distance. If the object is not at the desired distance, the electronic device may output an alert or other indication until the object is placed at the desired distance. For example, the electronic device may provide the user with user interface display instructions, audio interactions, or other feedback so that the user can properly adjust the position of the object relative to the UV light source.

In some implementations, the object is oriented at a desired orientation relative to the UV light source of the electronic device. The orientation of the object may be ascertained using the camera. Not only is the object positioned at an optimal distance for UV exposure, but the object may be oriented in a manner to optimize surface area coverage for UV exposure. The electronic device may be configured to output instructions to the user for orienting the object at the desired orientation.

At the desired distance and orientation, the electronic device can determine an amount of UV intensity and an amount of surface area that is exposed to UV light by the UV light source. Given that intensity of UV light decreases over distance and the intensity distribution may vary over a given surface area, adequate disinfection of the object depends at least on optimal placement and orientation of the object relative to the UV light source. In some implementations, the desired distance may be predetermined by the electronic device using the data associated with the UV light source (e.g., data sheets). In some implementations, the desired orientation may be determined to optimize surface area coverage in view of the camera.

The wavelength may be selected depending on a distance between the object and the electronic device. Where the wavelength emitted by the UV light source is tunable, the desired distance may be within a range of distances. If the object is closer to the electronic device, then a lower wavelength may be selected to ensure safe and effective disinfection. If the object is farther from the electronic device, then a higher wavelength may be selected to ensure safe and effective disinfection.

In some implementations, the image of the object captured by the camera may be segmented into a plurality of segments. Each of the segments may correspond to different portions (e.g., first portion, second portion, etc.) of the object. Each of the segments may be represent an area that can be covered effectively by UV light at the desired distance. In some implementations, the plurality of segments may be an M×N matrix of segments.

FIG. 5B shows the image capture device of FIG. 5A positioned at a proper distance from the object to initiate disinfection of the object. The image capture device 500 obtains the image 520 of the object so that the image 520 is displayed in the user interface. After the object is identified by the image capture device 500 in FIG. 5A, the application determines an optimal distance or optimal range of distances between the object and the image capture device 500. The optimal distance or optimal range of distances may be calculated based on information regarding UV light emitted from the UV light source of the image capture device 500. Such information may be provided from a data sheet associated with the UV light source. Such information may include but is not limited to peak wavelength, irradiance, UV dose (fluence), scattering or intensity distribution, irradiation pattern, beam width, and viewing angle.

Assisted by the information from the data sheet, it is possible to calculate UV dose. The UV dose may correlate with an estimated reduction in number of live organisms for disinfection purposes. When emitting far UVC light at a wavelength of 220 nm, the far UVC light may destroy or inactivate microorganisms. As shown in Table 1, the percentage of microorganisms destroyed or inactivated on a given surface area depends on the UV dose.

TABLE 1

| Dose (mJ/cm²) | Reduction in number of live microorganisms |
|---|---|
| 5.4 | 99.0% |
| 10.8 | 99.0% |
| 16.2 | 99.9% |
| 21.6 | 99.99% |
| 27.0 | 99.999% |

The UV dose may be calculated according to the formula: H=E·t, where H corresponds to UV dose in mJ/cm², E corresponds to irradiance in mW/cm², and t corresponds to irradiation time in seconds. Thus, it is possible to reach the same dose with longer time and lower irradiance, and shorter time and higher irradiance. Irradiance (E) is inversely proportional to the distance (r) between the UV light source and the object. Irradiance (E) is directly proportional to the power (P) radiated by the UV light source. Accordingly, depending on the desired reduction in microorganisms, the UV dose (H) calculated from irradiance (E) and minimum exposure time (t) can determine an optimal distance for placement of the object.

With the optimal distance or optimal range of distances predetermined by the UV light source of the image capture device 500, the application may instruct the user via the user interface to position the image capture device 500 and the object at the optimal distance. Using the camera and/or depth sensor of the image capture device 500, a distance between the image capture device 500 and the object can be measured. Once the optimal distance is reached, the application may provide instructions 512 in the user interface indicating that the optimal distance has been reached and requesting that the user initiate disinfection of the object. If the optimal distance is not yet reached, the application may provide an alert or other signal to the user indicating that the optimal distance has not been reached.

If the optimal distance is not reached but the user chooses to initiate disinfection, the image capture device 500 may select a wavelength between prescribed ranges to achieve its functionality. Generally speaking, the UV light source may emit UV radiation at a wavelength between prescribed ranges (e.g., 207-222 nm or 207-220 nm for far UVC light). If the object is too close to the image capture device 500, the user may manually select or the application may automatically select a wavelength at a lower wavelength to ensure disinfection happens with a minimal wavelength prescribed. Or, if the object is too far from the image capture device 500, the user may manually select or the application may automatically select a wavelength at a higher wavelength to ensure disinfection happens with a maximum wavelength prescribed. In some implementations, the optimal distance may be a range of distances. The range of distances may correlate with the prescribed wavelength ranges of the UV light source that can still achieve a desirable level of disinfection of the object (e.g., 99.0% or greater).

In some implementations, the user may manually identify or the application may automatically identify an object for disinfection in the image 520. In some implementations, based on the object type, the application may determine if there is any risk related to exposing the object to UV light. For example, the image capture device 500 may provide a warning to the user or disable the UV light source if there is a risk associated with exposing the object to UV light. It is also possible that other objects in view of the camera may be identified by the application and deemed risky or dangerous for exposure to UV light.

After the object for disinfection is identified in the image 520, the image 520 may be segmented. How the image 520 is segmented may depend on the dimensions of the object and/or the beam coverage area of the UV light. The UV light may be emitted at a certain beam width or beam coverage area. Knowing UV dose and distance from the object, beam coverage area can be defined with a given UV dose. The beam coverage area can be defined as an area covered in a specific time frame. In some implementations, the beam width or beam coverage area may change with distance from the object. By way of an example, a focused beam may have a beam coverage area of 2 cm×2 cm (4 cm²) or 1 cm×1 cm (1 cm²) at the optimal distance. In some implementations, the beam coverage area may represent an area of UV irradiation that achieves a desirable level of disinfection for a certain exposure duration. The image 520 may be segmented into a plurality of segments 540, where each of the segments 540 may correspond to the beam coverage area at the distance between the object and image capture device 500. In some implementations, the image 520 may be segmented into an M×N array of segments 540. The application may convert the image 520 into the M×N array of segments 540 with an approximate time for sanitizing the object and a time interval for sanitizing each segment 540. Each segment 540 may be indicated with a marker, color, or other signal that specifies whether the segment 540 has been sanitized or not. For instance, each segment 540 is greyed out or saturated in a specific color to indicate that the segment has not been sanitized with UV light. In some implementations, the application may request that the camera of the image capture device 500 be positioned so that UV disinfection starts from a first row and first column of the M×N array of segments 540. However, it will be understood that UV disinfection may start at any row and column that the object is in.

Returning to FIG. 4, at block 430 of the process 400, a first portion of the object is exposed to UV light from the UV light source, where the object is at the desired distance from the UV light source. In some implementations, the UV light source is activated after the object is positioned at the desired distance. Activation of the UV light source may be initiated by the user or automatically by the electronic device. In some implementations, the UV light source is deactivated or disabled if the object or the first portion thereof is deemed risky for UV exposure. In some implementations, the electronic device outputs an alert or warning signal indicating that UV exposure of the object or the first portion thereof is deemed risky. Though far UVC light is generally harmless to humans, it is possible that the selected wavelength may be harmful to certain objects.

After the object is identified and positioned at the desired distance from the electronic device, at least some or all of the object in view of the electronic device is exposed to UV light. How much of the object is exposed to the UV light and for how long may be based on a beam width or beam coverage area of the UV light at the positioned distance from the electronic device. The beam width or beam coverage area may be based on the data associated with the UV light source.

In some implementations, the first portion of the object is exposed to the UV light for a first exposure duration. The first exposure duration may correspond to a duration for achieving a first UV dose, where the first UV dose reduces microorganisms at the first portion by a desirable amount (e.g., 99.0% or greater). The first exposure duration may be determined by the electronic device using the data associated with the UV light source (e.g., data sheet). Based on the correlation between UV dose and exposure duration, the exposure duration may depend on factors such as area, distance, and UV intensity. Longer exposure times increase the UV dose and shorter exposure times decrease the UV dose. In some implementations, if the first portion of the object is exposed to a duration exceeding an acceptable limit, then the UV light source may deactivate.

As discussed above, the image of the object may be segmented into a plurality of segments. Each segment of the image may correspond to an object area covered by the UV light. In other words, a focused beam of the UV light may have a beam coverage area that exposes the object area. The object area may be the first portion of the object. For instance, the first portion of the object may correspond to a first row and a first column (or other particular row and column) of the image in the M×N array of segments. The UV light is deemed to disinfect the first portion of the object after the first exposure duration as shown in one of the segments of the image.

The electronic device may include a flashlight configured to emit visible light. In some implementations, the flashlight is configured to emit visible light on the first portion of the object when the first portion is exposed to the UV light. This means that the object area covered by the UV light can be illuminated by the flashlight. This allows the user to visually track UV exposure on the object, thereby improving user perception of UV sanitization of the object.

The electronic device may output an indication that the first portion of the object has been disinfected. In some implementations, the electronic device may provide an indication to the user by visual, audio, or haptic feedback that the first portion of the object has been disinfected. For example, the segment of the image may change color or otherwise specify that the segment associated with the first portion has been disinfected. The indication may be provided after the first portion is exposed to the UV light for the first exposure duration. In some implementations, a progress of disinfecting the first portion or an entirety of the object may be indicated by a percentage, status bar, color change, or other form of progress tracking.

FIG. 5C shows the image capture device of FIG. 5B after multiple segments of the image are indicated as sanitized. When the object is positioned at the optimal distance and an image 520 is segmented, the application may activate the UV light source. Activation of the UV light source may occur via user input or automatically upon positioning the object at the optimal distance. The UV light source may expose an object area corresponding to at least one of the segments 540 to UV light. The duration of exposure may be sufficient to disinfect the object area.

The exposure duration to achieve sanitization of a segment 540 can be determined by the UV dose for achieving the desired level of disinfection, where the UV dose can be calculated based in part from the data sheet associated with the UV light source. In some implementations, where the desired level of sanitization is not met, the user interface may indicate how much of the segment 540 or the image 520 has been disinfected in terms of a percentage, status bar, color change, or other form of progress tracking. If the duration of UV exposure was not sufficient or the object was placed out of view from the camera during UV exposure, then sanitization of one or more segments 540 may be incomplete. If the duration of the UV exposure exceeds the desired exposure duration to achieve sanitization beyond an acceptable limit, then an alert may be provided or the UV light source is deactivated.

The image capture device 500 or the object may be moved relative to one another so that different areas of the object are exposed to UV light. Movement speed may be adequate to expose each object area to UV light for sufficient duration. As disinfection of the object proceeds in FIG. 5C, some of the segments 540 may become sanitized segments 550 and some of the segments 540 may remain as unsanitized segments 545. When an object area is exposed to UV light for the sufficient duration to achieve the desired level of disinfection, the segment 540 of the image 520 associated with the object area is changed to a sanitized segment 550. For instance, the sanitized segment 550 is no longer greyed out or saturated to a different color than the unsanitized segment 545. In FIG. 5C, the sanitized segments 550 include all rows of segments 540 in the first column and the second column of the M×N array of segments 540, and the unsanitized segments 545 include all rows of segments 540 in the third column of the M×N array of segments 540. In some implementations, where the UV light source is an array or bar of UV LEDs, multiple segments 540 may be sanitized at a time.

In some implementations, the application may guide the user to move the image capture device 500 relative to the object so that additional segments 540 are exposed to UV light for disinfection. As an analogy, the image capture device 500 acts like a brush, the UV light acts as paint, and the object acts like a canvas. In some other implementations, the application may guide the user to move the object relative to the image capture device 500 so that additional segments are exposed to UV light for disinfection. Instructions 514 provided in the user interface may instruct the user to move the image capture device 500 or object in a specified direction. The instructions 514 direct the user to navigate the image capture device 500 or object in a manner to cover the M×N array of segments 540. In some instances, the instructions 514 may further direct the user to perform one or more of the following: move the image capture device 500 or object at a specified speed, focus on an unsanitized segment 545 for a specified time, indicate a direction of movement, maintain a certain distance from the object, reposition or reorient the object, and indicate an unsanitized segment 545 to disinfect next. If the user does not follow the instructions 514, the image capture device 500 may deactivate the UV light sensor or prescribe new instructions to the user.

In some implementations, the segment 540 being covered for disinfection by UV light may be spotlighted or highlighted in the user interface by the application. In some implementations, a spot area, circle, or other visual indicator may be provided on the user interface to convey to the user where the UV exposure is occurring. In some implementations, a flashlight in the image capture device 500 may emit visible light on the object area being exposed to UV light to provide further visualization to the user where the UV exposure is occurring.

FIG. 5D shows the image capture device of FIG. 5C after more segments of the image are indicated as sanitized. Unsanitized segments 545 in FIG. 5C become sanitized segments 550 in FIG. 5D. In some implementations, portions of the image 520 that do not cover any part of the object may be blacked out or otherwise indicated as unneeded for disinfection. The image capture device 500 or the object is moved so that additional segments 540 are exposed to UV light and sanitized. After following instructions 514 in FIG. 5C, new instructions 516 in FIG. 5D may direct the user to segments 540 for object areas that have not been disinfected. The new instructions 516 are provided in the user interface to instruct the user to move the image capture device 500 or object in a specified direction. The new instructions 516 are provided to complete disinfection of the entire object.

By decomposing or segmenting the image 520 into a series of segments 540, sanitization may be done sequentially. The image capture device 500 may be moved in a simple manner such as up-down or left-right to achieve disinfection. Visual or audio interactions may guide the user. Moreover, the image capture device 500 may readily display a progress of disinfection on the user interface.

Returning to FIG. 4, at block 440 of the process 400, a user associated with the electronic device is optionally instructed to place the object relative to the camera so that at least a second portion of the object is positioned to be exposed to the UV light source. Based on identification of the object and/or selection of the object in view of the camera, it may be determined by the electronic device whether the entirety of the object has been disinfected or not. If not, one or more unsanitized portions adjacent to the first portion may be identified by the electronic device. The one or more unsanitized portions may include the second portion of the object.

Data regarding sanitization of the first portion of the object may be stored in the memory or database associated with the electronic device. Such data can include, for example, the identity of the object, what percentage or amount was sanitized, the image of the object, the time and date, portions that remain unsanitized, etc. That way, the user can resume sanitization for a remainder of the object if sanitization was terminated. Termination may result, for example, if a higher priority call or alert occurred in the electronic device, or if the object was removed from the view of the camera. The UV light source may be deactivated upon termination. Resuming sanitization of an object may require user authentication to the electronic device. Alternatively, the user can re-sanitize the object if too much time has elapsed since an earlier sanitization. The data regarding sanitization can be saved in the electronic device to track what objects have been sanitized and how much was sanitized.

After identifying the second portion of the object as unsanitized, instructions are provided to the electronic device via visual, auditory, or haptic feedback. In some implementations, the instructions are provided in a user interface of the electronic device. In some implementations, the instructions are provided as auditory commands from a speaker of the electronic device. The instructions may include, for example, where to position the object relative to the camera, a direction of movement, how much time to expose the second portion to UV light, and a distance to maintain from the object, among other possible instructions. The electronic device may detect if the user has followed the instructions. If the user has not followed the instructions, the electronic device may deactivate the UV light source or prescribe new instructions. If the user has followed the instructions, the electronic device may activate the UV light source or keep the UV light source activated.

In some implementations, adjustments to the UV light source may be made by the user or automatically if conditions between the first portion and the second portion have changed. Example conditions that may change include a distance between the object and the electronic device, an orientation of the object, or a new object in view that is deemed risky or dangerous. The UV light source may emit a different wavelength or adjust its UV intensity depending on the conditions of the second portion of the object. The UV sensor coupled to the UV light may adjust the UV intensity.

At block 450 of the process 400, at least the second portion of the object is optionally exposed to UV light from the UV light source. The UV light source may be re-activated after the object is positioned at the desired distance or the UV light source may be kept activated from previously exposing the first portion of the object. In some implementations, the UV light source is deactivated or disabled if the object or the second portion thereof is deemed risky for UV exposure. In some implementations, the electronic device outputs an alert or warning signal indicating that UV exposure of the object or the second portion thereof is deemed risky.

In some implementations, the second portion of the object is exposed to the UV light for a second exposure duration. The second exposure duration may correspond to a duration for achieving a second UV dose, where the second UV dose reduces microorganisms at the second portion by a desirable amount (e.g., 99.0% or greater). The second exposure duration may be determined by the electronic device using the data associated with the UV light source (e.g., data sheet). Based on the correlation between UV dose and exposure duration, the exposure duration may depend on factors such as area, distance, and UV intensity. In some implementations, if the second portion of the object is exposed to a duration exceeding an acceptable limit, then the UV light source may deactivate.

The electronic device may output an indication that the second portion of the object has been disinfected. The indication may be provided after the second portion is exposed to the UV light for the second exposure duration. In some implementations, a progress of disinfecting the second portion or the entirety of the object may be indicated by a percentage, status bar, color change, or other form of progress tracking.

At block 460 of the process 400, the object is determined to have been disinfected. In some implementations, the operations at blocks 440 and 450 may be repeated on additional portions or surfaces of the object until the entirety of the object is disinfected. Thus, exposure to UV light may occur on a third portion, fourth portion, fifth portion, and so on for the object. The object is continually positioned relative to the electronic device so that additional portions are disinfected by UV light. After all portions or surfaces of the object are exposed to UV light for a sufficient duration, the object is deemed disinfected or sanitized. As discussed above, the image of the object may be segmented into a plurality of segments. When each of the segments associated with the object has been exposed for a sufficient duration, disinfection is completed for the object.

Having identified the object and its dimensions, the electronic device can track the progress of disinfecting the object. In some implementations, the electronic device can provide an indication to the user via visual, auditory, or haptic feedback of how much the object has been disinfected. Once the electronic device determines that a threshold amount of the object has been disinfected by UV light, the electronic device may provide an indication of successful completion via visual, auditory, or haptic feedback.

In some implementations, the object that has been disinfected may be stored in the memory or other database associated with the electronic device. The memory or database may maintain a list of objects that has been sanitized and when. Accordingly, it can be remembered when objects such as a left hand, a right hand, a table, vegetables, glasses, a chair, television, or T-shirt were sanitized and how frequently. In some implementations, the information in the memory or database can be used for analytics or statistical studies. In some implementations, such information can be used to alert or remind the user to perform sanitization, or such information can be presented visually to the user. In some implementations, the information stored in the memory or database can be linked with data stored in a service platform such as a national health application. It's possible that the data stored in the service platform can be used to communicate to the user if contact has been made with a person who has an infectious disease like Covid-19. The user may be alerted to sanitize himself or surrounding areas to limit the spread of the infectious disease.

FIG. 5E shows the image capture device of FIG. 5D after sanitization is complete. After all the segments 540 associated with the object are sanitized to become sanitized segments 550 in the image 520, the application may determine that sanitization of the object is complete. The application may give an indication 518 in the user interface signaling that the object has been successfully disinfected. Information regarding the sanitized object, such as the identity of the object and when the object was sanitized, may be stored in a database associated with the image capture device 500. This information may be retrieved later by the user or compiled in a statistical study.

Though the object for disinfection in FIGS. 5A-5E was a person's hand, it will be understood that any animate or inanimate object may be subject to UV disinfection in the present disclosure. Such objects may even include fields, crops, roads, buildings, drinking fountains, and other common places. Though the image capture device 500 shown in FIGS. 5A-5E was a smartphone, it will be understood that any electronic device configured to capture images may be used for UV disinfection. For instance, a drone may be employed to disinfect fields, crops, roads, buildings, drinking fountains, and other common places. In such instances, the drone may employ a UV light source at higher wavelengths other than UVC radiation.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor or any conventional processor, controller, microcontroller or state machine. A processor may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, the structures disclosed in this specification and their structural equivalents thereof, or in any combination thereof. Implementations of the subject matter described in this specification may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module that may reside on a computer-readable medium. Computer-readable media include both computer storage media and communication media including any medium that may be enabled to transfer a computer program from one place to another. Storage media may be any available media that may be accessed by a computer. By way of example and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein.

Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

What is claimed is:

1. An electronic device comprising:
an imaging source;
an ultraviolet (UV) light source; and
a control system communicatively connected to the imaging source and the UV light source, wherein the control system is configured to:
identify an object for disinfection using the imaging source;
expose at least a first portion of the object to UV light from the UV light source, wherein the object is at a desired distance from the UV light source; and
determine that the object has been disinfected.

2. The electronic device of claim 1, further comprising:
a display, wherein the imaging source is configured to display an image showing the object to be disinfected in the display.

3. The electronic device of claim 2, wherein the control system is further configured to:
segment the image containing the object into a plurality of segments, each segment corresponding to different portions of the object; and
expose each portion of the object corresponding to the plurality of segments to UV light for a sufficient duration to complete disinfection of the object.

4. The electronic device of claim 1, wherein the UV light source is configured to emit far UVC light.

5. The electronic device of claim 1, wherein the desired distance is calculated based at least in part on an intensity of the UV light, a wavelength of the UV light, and desired level of disinfection in at least the first portion of the object.

6. The electronic device of claim 1, wherein the control system is further configured to:
instruct a user associated with the electronic device to place the object relative to the imaging source so that at least a second portion of the object is positioned to be exposed to the UV light source; and
expose at least the second portion of the object to UV light from the UV light source.

7. The electronic device of claim 1, wherein the control system is further configured to:
provide an indication to a user via visual, auditory, or haptic feedback of how much the object has been disinfected.

8. The electronic device of claim 1, wherein the control system is further configured to:
provide an indication to a user associated with the electronic device based on the object being at the desired distance.

9. The electronic device of claim 1, wherein the control system configured to expose at least the first portion of the object to UV light is configured to expose at least the first portion of the object to UV light for a specified duration to deliver a desired level of UV dose of UV light.

10. The electronic device of claim 1, wherein the control system is further configured to:
deactivate the UV light source based on the object not being in view of the imaging source.

11. The electronic device of claim 1, further comprising:
a flashlight, wherein the flashlight is configured to emit visible light on at least the first portion of the object exposed to the UV light.

12. The electronic device of claim 1, wherein the electronic device is a smartphone, the imaging source is a camera, and the UV light source includes one or more UV light emitting diodes (LEDs) configured to emit a wavelength between about 207 nm and about 222 nm.

13. A method for disinfecting an object, the method comprising:
identifying an object for disinfection using a camera of an electronic device, wherein the electronic device includes the camera and a UV light source;
exposing at least a first portion of the object to UV light from the UV light source, wherein the object is at a desired distance from the UV light source; and
determining that the object has been disinfected.

14. The method of claim 13, further comprising:
instructing a user associated with the electronic device to place the object relative to the camera so that at least a second portion of the object is positioned to be exposed to the UV light source; and
exposing at least the second portion of the object to UV light from the UV light source.

15. The method of claim 13, further comprising:
providing an indication to a user via visual, auditory, or haptic feedback of how much the object has been disinfected.

16. The method of claim 13, wherein exposing at least the first portion of the object to UV light includes exposing at least the first portion of the object to UV light for a specified duration to deliver a desired level of UV dose.

17. The method of claim 13, wherein the electronic device further includes a display for displaying an image showing the object to be disinfected, wherein the method further comprises:
segmenting an image containing the object into a plurality of segments, each segment corresponding to different portions of the object; and
exposing each portion of the object corresponding to the plurality of segments to UV light for a sufficient duration to complete disinfection of the object.

18. The method of claim 13, further comprising:
providing an indication to a user associated with the electronic device based on the object being at the desired distance.

19. The method of claim 13, wherein the electronic device further includes a flashlight, wherein the method further comprises:
emitting visible light from the flashlight corresponding to at least the first portion of the object exposed to the UV light.

20. The method of claim 13, wherein the UV light has a wavelength between about 207 nm and about 222 nm.
